(19) United States
(12) Patent Application Publication
(10) Pub. No.: US 2014/0379276 A1
(43) Pub. Date: Dec. 25, 2014

(54) METHOD FOR CALCULATING NITROGEN CONCENTRATION IN SILICON SINGLE CRYSTAL AND METHOD FOR CALCULATING RESISTIVITY SHIFT AMOUNT

(71) Applicants: Ryoji Hoshi, Nishishirakawa-gun (JP); Hiroyuki Kamada, Nishishirakawa-gun (JP)

(72) Inventors: Ryoji Hoshi, Nishishirakawa-gun (JP); Hiroyuki Kamada, Nishishirakawa-gun (JP)

(73) Assignee: SHIN-ETSU HANDOTAI CO., LTD., Tokyo (JP)

(21) Appl. No.: 14/241,255

(22) PCT Filed: Aug. 8, 2012
(86) PCT No.: PCT/JP2012/005024

§ 371 Date: Feb. 26, 2014

(30) Foreign Application Priority Data
Sep. 8, 2011 (JP) 2011-195845

(51) Int. Cl. G01N 27/14; G01N 27/04; H01L 29/167; G01N 33/00

(52) U.S. Cl. CPC G01N 27/14; G01N 33/00; G01N 27/041; H01L 29/167; G01N 2033/0095
USPC 702/23

(57) ABSTRACT

A method for calculating a nitrogen concentration in a silicon single crystal doped with nitrogen, wherein the correlation among a carrier concentration difference Δ[n] obtained from a difference between resistivity after heat treatment by which an oxygen donor is eliminated and resistivity after heat treatment by which a nitrogen-oxygen donor is eliminated, an oxygen concentration [Oi], and a nitrogen concentration [N] in the nitrogen-doped silicon single crystal is obtained in advance, and an unknown nitrogen concentration [N] in a nitrogen-doped silicon single crystal is obtained by calculation from the carrier concentration difference Δ[n] and the oxygen concentration [Oi] based on the correlation. As a result, a method for calculating a nitrogen concentration in a silicon single crystal, the method that can obtain the value of a nitrogen concentration even when an oxygen concentration is different, and a method for calculating the shift amount of resistivity are provided.

### TECHNICAL FIELD

The present invention relates to methods for calculating a nitrogen concentration and methods for calculating a resistivity shift amount in a nitrogen-doped silicon single crystal and, in particular, to a method for calculating a nitrogen concentration and a method, for calculating resistivity shift amount in a nitrogen-doped silicon single crystal grown by the Czochralski method (the CZ method).

### BACKGROUND ART

In production of a silicon single crystal, doping with nitrogen is sometimes performed to control a crystal defect or control oxygen precipitates called BMDs. In an FZ crystal or the like, the doping amount sometimes reaches a doping amount with a nitrogen concentration on the order of the 14th or 15th power of 10, but, in a CZ crystal, in particular, it has been reported in various documents that an adequate effect is produced even when a nitrogen concentration is 1×10¹⁴/cm³or less.

As a method for measuring the concentration of doping nitrogen, secondary ion mass spectroscopy (SIMS) is effective as local analysis, but the detection sensitivity thereof is in the middle of the order of the 14th power of 10, and measurement is impossible in concentrations of 1×10¹⁴/cm 3 or less. As a simpler and more sensitive method, Fourier trans form infrared spectroscopy (FT-IR) or the like is used.

These nitrogen concentration measurement methods are well summarized in Non-patent Literature 1. Nitrogen in silicon is described as taking various forms such as NN, NNO or NNOO. Absorption of an infrared region in vibration modes in these various forms is generally measured by FT-IR. It is reported that these forms change according to a treatment temperature. By increasing sensitivity by observing all of these various absorption peaks or removing background noise generated by a donor caused by oxygen (an oxygen donor) as in Patent Literature 1, an attempt to increase the detection sensitivity has been made. Non-patent Literature 1 pieces together various measurement techniques and reports that the detection sensitivity of infrared absorption by NN, NNO, and NNOO is 1×10¹⁴ atoms/cc.

As a method for obtaining a concentration that is lower than that, Patent Literature 2 focuses attention on the fact that nitrogen forms a donor and obtains a nitrogen concentration based on a change in resistivity caused when a donor caused by nitrogen is formed by 500 to 800° C. heat treatment after a donor caused by nitrogen (a nitrogen-oxygen donor) is eliminated by heat treatment at 1000° C. or more.

In Non-patent Literature 2 and Patent Literature 3, a nitrogen-oxygen donor in a low nitrogen concentration region is disclosed in more detail. Here, it is reported that nitrogen takes a different form ONO, not the forms NN, NNO, and NNOO described above, when a nitrogen concentration is 1×10 ¹⁴/cm³ or less and ONO acts as a donor.

In Non-patent Literature 2 and Patent Literature 3, though not a simple method, a nitrogen-oxygen donor amount is measured, by far infrared absorption at an extremely low temperature (liquid He temperature). Since the ratio between a nitrogen concentration and a nitrogen-oxygen donor is 1:1 when the nitrogen concentration is 1×10¹⁴/cm ³ or less, quantitative measurement of a nitrogen concentration may be possible by applying this technique.

In addition, in Patent Literature 4, a method for obtaining a nitrogen concentration from the state of a defect is proposed. As a defect, a Grown-in defect, a BMD, and the like are described.

Patent Literature 1: Japanese Unexamined Patent Publication (Kokai) No. 2003-240711

Patent Literature 2: Japanese Unexamined Patent Publication (Kokai) No. 2000-332074

Patent Literature 3: Japanese Unexamined Patent Publication (Kokai) No. 2004-111752

Patent Literature 4: Japanese Unexamined Patent Publication (Kokai) No. 2002-353282

Non-patent Literature 1: JEITA EM-3512

Non-patent Literature 2: K. Ono and M. Horikawa Jpn. J. Appl. 42 (2003) L261

### DISCLOSURE OF INVENTION

### Problem to be Solved by the Invention

As described above, as the method for obtaining a nitrogen concentration, there are Patent Literatures 1 to 4 and so forth.

However, as described in V. V. Voronkov et al. J. Appl. Phys. 89 (2001) 4289 and so forth, it is known that a donor caused by nitrogen is a nitrogen-oxygen donor (hereinafter, also referred, to as an NO donor) which is also associated with oxygen. Therefore, the concentration of nitrogen-oxygen donors is supposed to depend not only on nitrogen but also on an oxygen concentration.

Thus, the method of Patent Literature 2 cannot be used as it is when an oxygen concentration is different and is supposed to require a calibration curve for each oxygen concentration as described in Patent Literature 2, and therefore it cannot be said that the method has general versatility.

Moreover, as for Non-patent Literature 2 and Patent Literature 3, it can also be imagined that there is nitrogen that cannot form a nitrogen-oxygen donor due to an insufficient oxygen concentration when an oxygen concentration changes greatly and, for example, becomes a low oxygen concentration.

In these documents, it is imagined that the reason why a nitrogen concentration and a nitrogen-oxygen donor are shifted from a correlation of 1:1 when a nitrogen

concentration is 1×10¹⁴/cm³ or more is that nitrogen that cannot form a nitrogen-oxygen donor forms NN and so forth described above. That is, it is estimated that, even when the techniques disclosed in these documents are applied, an accurate nitrogen concentration cannot be obtained if an oxygen concentration is different.

Furthermore, also in Patent Literature 4, it is known that a situation in which a Grown-in defect or a BMD defect occurs also depends on an oxygen concentration. BMD is an abbreviation of Bulk Micro Defect and means an oxygen precipitate. The crystal defects such as a BMD and an OSF (Oxygen induced Stacking Fault) are defects associated with oxygen, and it is known that these defects becomes larger and dense when an oxygen concentration is high. As for Grown-in defect, a defect called a Void defect is said to have an oxide film (an inner-wall oxide film) inside the defect, and our findings reveal that the density thereof also depends on an oxygen concentration. However, in Patent Literature 4, quantitative examinations of the degree of influence of an oxygen concentration have not been made.

As described above, in the existing techniques, to obtain a nitrogen concentration, in particular, a low nitrogen, concentration of 1×10¹⁴/cm³ or less, ways such as using a nitrogen-oxygen donor as an index or using a crystal defect as an index have been devised.

However, in these existing techniques, the degree of influence of an oxygen concentration is not mentioned, and there is a problem that a situation with a different oxygen concentration cannot be dealt with immediately.

Thus, the present invention has been made in view of the problems described above, and an object thereof is to provide a method for calculating a nitrogen concentration in a silicon single crystal, the method that can obtain the value of a nitrogen concentration even when an oxygen concentration is different. Moreover, an object is to provide a method for calculating the shift amount of resistivity by heat treatment by which a nitrogen-oxygen donor is eliminated.

### Means for Solving Problem

To achieve the object described above, the present invention provides a method for calculating a nitrogen concentration in a silicon single crystal doped with nitrogen, wherein a correlation among a carrier concentration difference Δ[n] obtained from a difference between resistivity after hear treatment by which an oxygen donor is eliminated and resistivity after heat treatment by which a nitrogen-oxygen donor is eliminated, an oxygen concentration [Oi], and a nitrogen concentration [N] in the nitrogen-doped silicon single crystal is obtained in advance, and an unknown nitrogen concentration [N] in a nitrogen-doped silicon single crystal is obtained by calculation from the carrier concentration difference Δ[n] and the oxygen concentration [Oi] based on the correlation.

With such a method, when an unknown nitrogen concentration in a nitrogen-doped silicon single crystal is obtained by using the carrier concentration difference, it is possible to perform calculation with dealing with nitrogen-doped silicon single crystals with various oxygen concentrations. Since the oxygen concentration is also taken into consideration, it is possible to obtain a nitrogen concentration more accurately than the existing method. In addition, a nitrogen concentration can be obtained easily because a nitrogen concentration can be obtained by calculation from the carrier concentration difference and the oxygen concentration based on the correlation obtained in advance.

At this time, it is possible that when the unknown nitrogen, concentration [N] is calculated, calculation is performed by using a correlation expression:

[N]=(Δ[n]−β)/α[Oi]^{2.5 to 3.5}

(where α and β are constants)
from the carrier concentration difference Δ[n] and the oxygen concentration [Oi].

As described above, calculation can be performed easily by using the correlation expression described above. Incidentally, the constants α and β can be determined as appropriate according to the measurement conditions such as the oxygen concentration.

Moreover, it is possible that the nitrogen-doped silicon single crystal is a nitrogen-doped silicon single crystal grown by a Czochralski method.

In a CZ crystal, even when a nitrogen concentration is, for example, a low nitrogen concentration of 1×10¹⁴/cm³ or less which is too low to be measured by SIMS or FT-IR, an adequate nitrogen doping effect is supposed to be able to be obtained. The present invention is effective when obtaining the nitrogen concentration of a CZ crystal that is regarded as being useful even when the nitrogen concentration thereof is low, not to mention a CZ crystal with a nitrogen concentration that can be measured by SIMS or the like. Moreover, since the CZ crystal contains a large amount of oxygen, the present invention that can perform a measurement by eliminating the influence thereof is effective.

Moreover, the present invention provides a method for calculating a resistivity shift amount in a silicon single crystal doped with nitrogen, wherein a correlation among a carrier concentration difference Δ[n] obtained from a difference between resistivity after heat treatment by which an oxygen donor is eliminated and resistivity after heat treatment by which a nitrogen-oxygen donor is eliminated, an oxygen concentration [Oi], and a nitrogen concentration [N] in the nitrogen-doped silicon single crystal is obtained in advance, and an unknown carrier concentration difference Δ[n] in a nitrogen-doped silicon single crystal is calculated from the nitrogen concentration [N] and the oxygen concentration [Oi] based on the correlation and a resistivity shift amount by the heat treatment by which the nitrogen-oxygen donor is eliminated is obtained from the calculated carrier concentration difference Δ[n].

With such a method, it is possible to calculate the carrier concentration difference, with dealing with nitrogen-doped silicon single crystals with various oxygen concentrations, more easily and accurately than the existing method and obtain the shift amount of resistivity by heat treatment by which a nitrogen-oxygen donor is eliminated. Furthermore, it is possible to obtain a resistivity shift amount without performing heat treatment for nitrogen-oxygen donor elimination.

At this time, it is possible that when the unknown carrier concentration difference Δ[n] is calculated, calculation is performed by using a correlation expression:

[n]=α[N]×[Oi]^{2.5 to 3.5}+β

(where α and β are constants)
from the nitrogen concentration [N] and the oxygen concentration [Oi].

As described above, calculation can be performed easily by using the correlation expression described above. Incidentally, the constants α and β can be determined as appropriate according to the measurement conditions such as the oxygen concentration.

Moreover, it is possible that the nitrogen-doped silicon single crystal is a nitrogen-doped silicon single crystal grown by a Czochralski method.

The present invention is effective because, in the present invention, it is possible to obtain the nitrogen concentration of a CZ crystal that contains a large amount of oxygen and is regarded as being useful even when the nitrogen concentration thereof is too low to be measured.

### Effect of the Invention

As described above, according to the present invention, it is possible to obtain a nitrogen concentration in a single crystal by calculation with dealing with nitrogen-doped silicon single crystals with various oxygen concentrations. Moreover, it is possible to obtain a resistivity shift amount caused by heat treatment for nitrogen-oxygen donor elimination. In addition, it is possible to obtain the nitrogen concentration and the resistivity shift amount more easily and accurately than the existing method.

### BRIEF DESCRIPTION OF DRAWINGS

FIG. 1 is a flowchart describing an example of steps of a method for calculating a nitrogen concentration in a silicon single crystal of the present invent ion;

FIG. 2 is a flowchart describing an example of steps of a method for calculating a resistivity shift amount of the present invention;

FIG. 3 is a graph depicting the relationship between a carrier concentration difference and a nitrogen concentration in a preliminary test in Example 1;

FIG. 4 is a graph depicting the relationship between the carrier concentration difference and an oxygen concentration in the preliminary test in Example 1; and

FIG. 5 is a graph depicting the relationship between the carrier concentration difference and the product of the first power of a nitrogen concentration and the third power of an oxygen concentration in the preliminary test in Example 1.

### BEST MODE FOR CARRYING OUT THE INVENTION

Hereinafter, an embodiment of the present invention will be described in detail with reference to the drawings, but the present invention is not limited thereto.

As described earlier, when an unknown nitrogen concentration in a nitrogen-doped silicon single crystal is obtained by using a carrier concentration difference obtained from the difference between resistivity after heat treatment by which an oxygen donor is eliminated and resistivity after heat treatment by which a nitrogen-oxygen donor is eliminated (hereinafter, also simply referred to as a carrier concentration difference), since the nitrogen-oxygen donor depends on an oxygen concentration, if the oxygen concentration changes, it is necessary to obtain a calibration curve for each oxygen concentration in snob a method as described in Patent Literature 2.

Thus, first, the correlation among the carrier concentration difference, the oxygen concentration, and the nitrogen concentration in a nitrogen-doped silicon single crystal is obtained in advance. The inventors of the present invention have found out that, by then obtaining the carrier concentration difference and the oxygen, concentration in a single crystal to be measured, the single crystal, whose nitrogen concentration is unknown, by measurement or the like and calculating the nitrogen concentration based on the above-described correlation, the nitrogen concentration can be obtained easily for various oxygen concentrations and have completed the present invention.

A method for calculating a nitrogen concentration in a silicon single crystal of the present invention will be described.

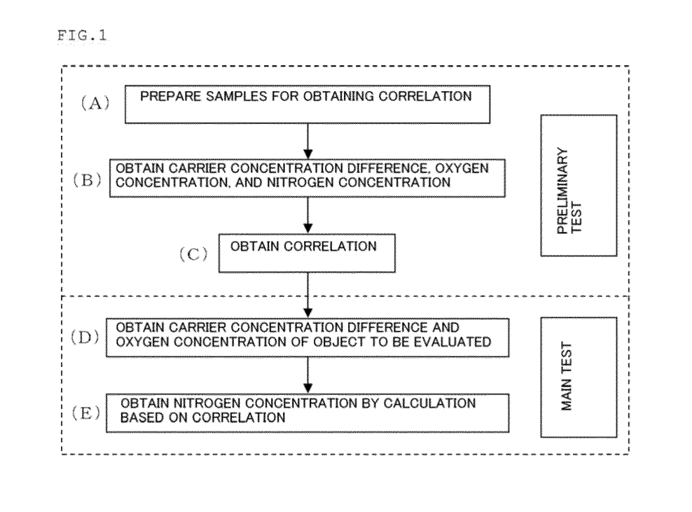

FIG. 1 is a flowchart describing an example of steps. The steps are broadly divided into a preliminary test and a main test. With the preliminary test, the correlation among a carrier concentration difference, an oxygen concentration, and a nitrogen concentration in a nitrogen-doped silicon single crystal is examined and obtained from samples for the preliminary test. Then, in the main, test, a carrier concentration difference and an oxygen concentration of a nitrogen-doped silicon single crystal (whose nitrogen concentration is unknown) which is an object to be evaluated are obtained, and these values are applied to the correlation obtained in the preliminary test, whereby the nitrogen concentration is calculated.

Hereinafter, the preliminary test and the main test will be described more specifically.

### Preliminary Test

### Samples for Obtaining a Correlation Are Prepared: FIG. 1(A))

First, samples for obtaining the correlation among a carrier concentration difference, an oxygen concentration, and a nitrogen concentration in a nitrogen-doped silicon single crystal are prepared.

The number of samples is not limited to a particular number and can be determined as occasion demands. Moreover, the ranges of a carrier concentration difference, an oxygen concentration, and a nitrogen concentration in each sample are not limited to particular ranges and can be determined in accordance with an expected value of a nitrogen concentration in a single crystal that is actually evaluated in the main test, for example. An appropriate number of samples in an appropriate range of each element can be prepared in order to obtain a more accurate nitrogen concentration in the main test.

Incidentally, here, descriptions will be given by taking up, as an example of a sample for the preliminary test and an object to be evaluated in the main test which will be described later, a silicon single crystal grown while being doped with nitrogen by the CZ method, but a method for producing a crystal used therefor is not limited to a particular method, and any crystal can be used as long as the crystal can obtain the correlation between the elements as a sample for the preliminary test.

Moreover, the growth of a crystal by the CZ method is not limited to particular growth and, for example, a method similar to the existing method can be adopted. Since a crystal produced by the CZ method contains a large amount of oxygen and is regarded as being useful even when the crystal is a crystal whose nitrogen concentration is too low to be measured by SIMS or the like, the present invention is especially effective in calculating the nitrogen concentration of such a CZ crystal.

### A Carrier Concentration Difference, an Oxygen Concentration, and a Nitrogen Concentration Are Obtained: FIG. 1(B))

Next, a carrier concentration difference, an oxygen concentration, and a nitrogen concentration of each of the prepared samples are obtained.

First, a way of obtaining the carrier concentration difference will be described.

This step is mainly formed of heat treatment by which an oxygen donor is eliminated, subsequent measurement of resistivity, furthermore, heat treatment by which a nitrogen-oxygen donor is eliminated, and subsequent measurement of resistivity. That is, an oxygen donor and a nitrogen-oxygen donor exist in a crystal of a nitrogen-doped silicon single crystal grown by the CZ method, the heat treatment by which the oxygen donor is eliminated is performed at a relatively low temperature as will be described later, the oxygen donor is eliminated from the crystal by the heat treatment, and. resistivity is measured. At this time, since the nitrogen-oxygen donor still remains in the crystal, the resistivity here is resistivity in a state in which no oxygen donor exists and the nitrogen-oxygen donor exists.

Next, the heat treatment by which the nitrogen-oxygen donor is eliminated is performed at a relatively high temperature, and the nitrogen-oxygen donor in the crystal is eliminated by the heat treatment. Therefore, it is possible to measure resistivity in a state in which neither the oxygen donor nor the nitrogen-oxygen donor exists.

In addition, from the difference in resistivity, it is possible to obtain a carrier concentration difference caused by the nitrogen-oxygen donor.

Here, the heat treatment for oxygen donor elimination and the heat treatment for nitrogen-oxygen donor elimination will be described more specifically.

Since the oxygen donor is generated in a relatively low-temperature region whose temperature is around 450° C., the bottom, side of the CZ crystal does not undergo such a low-temperature thermal history and almost no oxygen donor is generated on the bottom side. On the other hand, the top side of the crystal undergoes adequately this thermal history region, many oxygen donors are generated on the top side. As a crystal, has become longer recently, this tendency becomes increasingly pronounced, and a lot of oxygen donors exist, on the top side and almost no oxygen donor exists on the bottom side.

It is known that this oxygen donor is eliminated by mild heat treatment which is performed at 650° C. for about 20 minutes, for example. Various types of heat treatment by which an oxygen donor is eliminated have been proposed and there is, for example, high-temperature short-time heat treatment using RTA (Rapid Thermal Anneal); here, the temperature and the time of heat treatment are not limited to particular temperature and time and any heat treatment may be adopted as long as the heat treatment can eliminate an oxygen donor caused by oxygen.

Moreover, it is described that the nitrogen-oxygen donor disappears by relatively high-temperature heat treatment of, for example, 900° C. in Patent Literature 3, 1000° C. in Patent Literature 2, and 1050° C. in WO 2009/025337. Furthermore, it is described that the temperature at which the nitrogen-oxygen donor is generated is, for example, 500 to 800° C. in Patent Literature 2 and 600 to 700° C. in Patent literature 3 and the nitrogen-oxygen donor is generated at a high temperature compared to the oxygen donor. In addition, as described in Patent Literature 2, the generation amount is saturated by relatively short-time heat treatment. It Is for this reason that the nitrogen-oxygen donor is generated relatively uniformly as compared to the oxygen donor which is generated at high density on the top side of the crystal. Moreover, it cannot be said that the nitrogen-oxygen donor is not affected by a furnace structure and a growth rate that affect the thermal history of the grown crystal. However, the impact is relatively small and it is unlikely that the nitrogen-oxygen donor amount differs greatly depending on these growth conditions.

Based on those described above, by measuring resistivity after performing mild heat treatment at about 650° C., for example, as heat treatment for oxygen donor elimination, obtaining a carrier concentration calculated from the resistivity, then measuring resistivity after performing high-temperature heat treatment at 900° C. or more, for example, as heat treatment for nitrogen-oxygen donor elimination, and obtaining a carrier concentration calculated from the resistivity, it is possible to obtain a carrier concentration difference Δ[n] caused by the nitrogen-oxygen donor based on the difference of them. Here, Irvin curve may be used to obtain a carrier concentration from the resistivity.

Incidentally, the method for measuring resistivity is not limited to a particular method; for example, resistivity can be measured by a four-point probe method or the like.

Next, a way of obtaining an oxygen concentration will be described.

An oxygen concentration [Oi] can be obtained by, for example, FT-IR at ambient temperature. The reason why Oi is described in [Oi] is that an oxygen atom exists in an interstitial position in a silicon crystal and infrared absorption is measured in that position and is written as an oxygen concentration. Oxygen in which an oxygen atom forms an oxygen precipitate (BMD) as a result of oxygen precipitation heat treatment being performed does not cause absorption as [Oi], but the oxygen concentration mentioned here is naturally the oxygen concentration in a state in which the precipitation heat treatment is not performed.

When the sample has normal resistivity, FT-IR is used; however, when the sample is a low resistivity crystal, infrared light is absorbed, which makes it impossible to use FT-IR. Thus, an oxygen concentration is sometimes measured by a gas fusion method.

Incidentally, oxygen leaking out from a quartz crucible moves through silicon melt, most of the oxygen evaporates near the surface of the melt, and only an extremely small part of the oxygen is taken into the crystal. Therefore, since the oxygen concentration in the silicon crystal changes with various operation conditions, the oxygen concentration is generally measured and ensured by the above-described FT-IR or the like.

In any case, measurement of resistivity and measurement of an oxygen concentration are the most basic operations of assurance and evaluation of a CZ silicon and simple and versatile evaluation methods.

Moreover, an example of a way of obtaining a nitrogen concentration in the preliminary test will be described.

Doping with nitrogen in production of a CZ silicon single crystal is generally performed by a method by which a nitrogen doping agent is put into a crucible and is melted with a silicon raw material. As long as the initial amount of a doping agent is clear, the doping agent is introduced into a silicon crystal by segregation, which makes it possible to obtain a nitrogen concentration by calculation.

### A Correlation Is Obtained: FIG. 1(C))

After the carrier concentration difference, the oxygen concentration, and the nitrogen concentration of each sample are obtained in the manner described above, the correlation among them is obtained. A way of obtaining the correlation is not limited to a particular way, and any way may be adopted as long as the way can appropriately obtain the correlation among the carrier concentration difference, the oxygen concentration, and the nitrogen concentration described above.

Here, an example of the correlation among the carrier concentration difference Δ[n], the oxygen concentration [Oi], and. the nitrogen concentration [N], the correlation obtained based on the carrier concentration difference Δ[n], the oxygen concentration [Oi], and the nitrogen concentration [N] actually obtained by the study and analysis assiduously conducted by the inventors of the present invention will be described specifically.

By the study and analysis, the inventors of the present invention have found out that, as an especially important tendency, Δ[n] is proportional to the first power of [N] and about the. third power of [Oi].

As in the above-described steps, various samples in which a nitrogen concentration [N] and an oxygen concentration [Oi] were set were prepared, an oxygen donor was eliminated, and the carrier concentration difference Δ[n] was obtained from the resistivity before and after nitrogen-oxygen donor elimination. The analysis of these data revealed that, when the oxygen concentration [Oi] was fixed, the carrier concentration difference Δ[n] was proportional to the first power of the nitrogen concentration [N] and, when the nitrogen concentration was fixed, the carrier concentration difference Δ[n] was proportional to about the third power of the oxygen concentration [Oi]. This is the result indicating that the nitrogen-oxygen donor may be formed of one nitrogen atom and three oxygen atoms. The further analysis using various data revealed that the carrier concentration difference Δ[n] was proportional to the 2.5th to 3.5th power of the oxygen concentration [Oi]. A multiplier factor may be selected from 2.5 to 3.5 based on the data (the carrier concentration difference Δ[n], the oxygen concentration [Oi], and the nitrogen concentration [N]) in the preliminary test.

Based on the results described above, a correlation expression in which the carrier concentration difference Δ[n] is proportional to the product of the first power of the nitrogen concentration [N] and the 2.5 to 3.5th power of the oxygen concentration [Oi] was derived. That is,

Δ[n]=α[N]×[Oi]^{2.5 to 3.5}+β

(where α and β are constants).
In addition, from a modified form of the correlation expression, an expression for obtaining the nitrogen concentration [N] was completed. That is,

[N]=(Δ[n]−β)/α[Oi]^{2.5 to 3.5}

(where α and β are constants).

Incidentally, here, the constants α and β are constants determined by the measurement conditions. For example, the oxygen concentration is measured by FT-IR, and conversion into an oxygen concentration is performed based on the absorbance obtained by subtracting a reference from the absorption peak. At this time, the conversion factor differs depending on the reference, the measuring instrument, and the manufacturer. Therefore, even when measurement is performed on the same sample, the oxygen concentration differs depending on the conversion factor used. The same goes for the nitrogen concentration measurement; at present, the nitrogen concentration adopted among the manufacturers is not obtained by correlating the results, and, even when the nitrogen concentrations have seemingly the same value, there is a possibility that the concentrations are actually different. As for the measurement of resistivity, the measurement is simple and there is no difference among the manufacturers, but there is a fluctuating element such as a donor killer heat treatment condition.

Since the manufacturers use their respective fixed process steps, comparison of the absolute values of the numerical values used in one manufacturer can be performed. However, it is difficult to perform comparison of the absolute values between one manufacturer and the other manufacturer, for example, and comparison using a conversion factor is required.

Since Δ[n], [Oi], and [N] are Δ[n], [Oi], and [N] measured under these circumstances, the values of α and β can he determined in the fined process steps of one manufacturer, but there is a high possibility that α and β respectively take different values in the other process steps. Thus, here, regarding numerics as those depending on the process steps, a fixed value is not used, and. the numerics are defined only as constants.

Moreover, based on a hypothesis that an NO donor is formed of one nitrogen atom and three oxygen atoms, it is preferable that β is 0. However, in actuality, the correlation expression is a correlation expression including various measurement errors, for example, causes of error such as certain heat treatment by which an NO donor cannot be eliminated completely, therefore, the expression also assuming a case where β is not 0 is adopted here.

When a series of process steps conditions changes greatly such as a change in the conversion factor, a correlation is obtained again, and redetermination can be performed or a correction coefficient can be used, as needed.

### Main Test

### A Carrier Concentration Difference and an Oxygen Concentration of an Object to be Evaluated are Obtained: FIG. 1(D))

A nitrogen-doped silicon single crystal, grown by the CZ method, the nitrogen-doped silicon single crystal whose nitrogen concentration is unknown, the nitrogen-doped silicon single crystal which is an object to be evaluated, is prepared, and a carrier concentration difference and an oxygen concentration are obtained, by measurement or the like.

The carrier concentration difference and the oxygen concentration can be obtained here by a method similar to the method, in the preliminary test. Since a nitrogen concentration in the main test is calculated in a subsequent step based on the correlation among the carrier concentration difference, the oxygen concentration, and the nitrogen concentration obtained by the preliminary test, it is preferable to obtain the carrier concentration difference and the oxygen concentration by process steps similar to the process steps in the preliminary test. This makes it possible to obtain a more accurate nitrogen concentration by calculation.

### A Nitrogen Concentration is Obtained by Calculation Based on the Correlation: FIG. 1(E))

By using the correlation obtained by the preliminary test, here,

[N]=(Δ[n]−β)/α[Oi]^{2.5 to 3.5}

(where α and β are constants)
of the correlation expression described above and substituting the carrier concentration difference Δ[n]and the oxygen concentration [Oi] obtained in the previous step thereinto, the unknown nitrogen concentration [N] can be obtained by calculation.

With such a method for calculating a nitrogen concentration of the present invention, it is possible to deal with a change in the oxygen concentration [Oi] and calculate a nitrogen concentration with ease. In addition, in obtaining a nitrogen concentration, an oxygen concentration that affects the nitrogen concentration is taken into consideration, which makes it possible to calculate a more accurate nitrogen concentration.

Next, a method for calculating a resistivity shift amount of the present invention will be described.

The method for obtaining an unknown nitrogen concentration when an object to be evaluated is an object whose nitrogen concentration is unknown has been described above. Here, a method for obtaining the shift amount of resistivity by heat treatment by which a nitrogen-oxygen donor is eliminated in the case of a nitrogen-doped silicon single crystal with a known nitrogen concentration will be described.

With the method of the present invention, when a nitrogen concentration is known, by obtaining an oxygen concentration in a silicon single crystal, it is possible to calculate a carrier concentration difference caused by a nitrogen-oxygen donor in the grown crystal and furthermore obtain a resistivity shift amount.

Since an oxygen donor can be eliminated at a relatively low temperature as described earlier, it is customary to measure resistivity after eliminating the oxygen donor and use the resistivity as a guaranteed value.

However, in a nitrogen-doped crystal (wafer), although the presence of a nitrogen-oxygen donor is known, there is no specific rule about a method of guarantee thereof, and the resistivity measured after simply eliminating the oxygen donor is sometimes used as a guaranteed value.

In such a case, for example, if a wafer process or a device process includes heat treatment performed at 900° C. or more, a nitrogen-oxygen donor is eliminated, and a shift of a resistivity value occurs. That is, a value presented as a guaranteed value differs from a resistance value of a device and the like after the process steps, and a problem may be produced also in the operation of the device.

Therefore, in the case of a silicon crystal with a known nitrogen concentration, only by measuring an oxygen concentration, it is possible to make a trial calculation of a resistivity shift amount after a device.

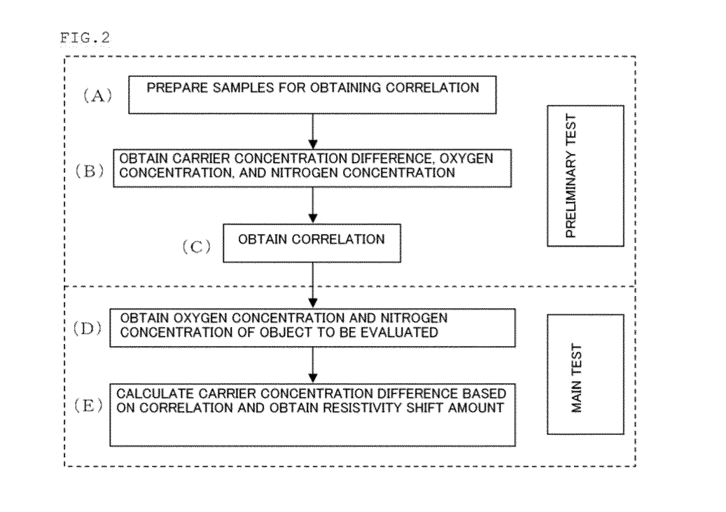

FIG. 2 is a flowchart describing an example of steps in the present invention. The steps are broadly divided into a preliminary test and a main test. By the preliminary test, from samples for the preliminary test, the correlation among a carrier concentration difference, an oxygen concentration, and a nitrogen concentration in a nitrogen-doped silicon single crystal are examined and obtained. Then, in the main test, for a nitrogen-doped silicon single crystal (whose carrier concentration difference is unknown) which is an object to be evaluated, the values of an oxygen concentration and a nitrogen concentration obtained by measurement or the like are applied to the correlation obtained by the preliminary test, whereby a carrier concentration difference is calculated, and a resistivity shift amount is then obtained.

Hereinafter, the preliminary test and the main test will be described more specifically.

A sample for obtaining a correlation is prepared: FIG. 2(A)), (A carrier concentration difference, an oxygen concentration, and a nitrogen concentration are obtained: FIG. 2(B)), and (A correlation is obtained: FIG. 2(C)) in the preliminary test can be performed in a manner similar to those of the method for calculating a nitrogen concentration in a silicon single crystal of the present invention, the method described with reference to FIG. 1. That is, as described earlier, for example, a correlation expression:

Δ[n]=α[N]×[Oi]^{2.5 to 3.5}+β

(where α and β are constants)
can be obtained.

Here, α and β are the same as those described earlier. Since the values thereof change depending on various measurement conditions as described earlier, it is preferable to use α and β determined under a particular condition as constant values. Unless there is a particular change, the values of α and β are the same as those obtained above. If the process step conditions change greatly such as a change in the conversion factor, redetermination can be performed or a correction coefficient can be used.

### Main Test

### An Oxygen Concentration and a Nitrogen Concentration of an Object to be Evaluated Are Obtained: FIG. 2(D))

A nitrogen-doped silicon single crystal grown by the CZ method, the nitrogen-doped silicon single crystal which is an object to be evaluated, is prepared, and an oxygen concentration and a nitrogen concentration are obtained. The oxygen concentration and the nitrogen concentration can be obtained here by a method similar to the method in the preliminary test.

### A Carrier Concentration Difference Is Calculated Based on the Correlation and a Resistivity Shift Amount Is Obtained: FIG. 2(E))

By using the correlation obtained by the preliminary test, here,

Δ[n]=α[N]×[Oi]^{2.5 to 3.5}+β

(where α and β are constants)
of the correlation expression described above and substituting the oxygen concentration [Oi] and the nitrogen concentration [N] obtained in the previous step thereinto, it is possible to calculate a carrier concentration difference Δ[n] caused by heat treatment by which a nitrogen-oxygen donor is eliminated.

By adding or subtracting the carrier concentration difference to or from the carrier concentration calculated from the resistivity after oxygen donor elimination, it is possible to calculate a resistivity shift amount by heat treatment for nitrogen-oxygen donor elimination and resistivity after the heat treatment. In addition, it is possible to deal with a change in the oxygen concentration and obtain them more easily and accurately as compared to the existing method. Incidentally, the reason why addition or subtraction is described here is that it depends on the conductivity type of an original silicon single crystal.

Moreover, for example, by determining the conditions of the heat treatment for nitrogen-oxygen donor elimination, the heat treatment simulating a heat treatment performed at 900° C. or more during a wafer process or a device process, it is possible to make a trial calculation of a resistivity shift amount after the device process or the like.

### EXAMPLES

Hereinafter, the present invention will be described, more specifically with an example and a comparative example, but the present invention is not limited to these examples.

### Example 1

A method for calculating a nitrogen concentration in a silicon single crystal in the present invention was performed.

First, a preliminary test was conducted, whereby the correlation among a carrier concentration difference, an oxygen concentration, and a nitrogen concentration was obtained.

Various nitrogen-doped silicon single crystal samples in which a target level of a nitrogen concentration was set at 3×10¹³ to 12×10¹³/cm³ and a level of an oxygen concentration was set at 2.5×10¹⁷ to 12×10¹⁷ atoms/cm³ (ASTM '79) were prepared.

These silicon single crystals which were samples for the preliminary test were grown by the CZ method.

In the CZ method, a quartz crucible filled with melt and a heater disposed so as to surround the crucible are provided. After a seed crystal is immersed in the crucible, a rod-like single crystal is pulled from the melt.

The crucible can move up and down in a crystal growth axis direction, and the crucible is moved upward in such a way as to compensate for a lowered liquid level of the melt reduced as s result of the melt having turned into a crystal during crystal growth. On the side of the crystal, an inert gas is made to flow to rectify the oxidizing steam generated from the silicon melt. Since the quartz crucible containing the melt is formed of silicon and oxygen, an oxygen atom dissolves in the silicon melt. The oxygen atom travels through the convection or the like in the silicon melt and eventually evaporates from the surface of the melt. At this time, most of the oxygen evaporates, but part of the oxygen is taken into the crystal and becomes interstitial oxygen Oi.

At this time, since it is possible to control the flow of the convection in the silicon melt by changing the revolution rate of the crucible or the crystal or changing the magnetic field application conditions in the magnetic field application CZ (MCZ) method and it is possible to control the amount of oxygen evaporation from the surface by adjusting the rate of flow of the inert gas or controlling the pressure inside a furnace, the oxygen concentration in the single crystal can be controlled.

By combining these control factors in various ways, samples whose levels of an oxygen concentration were set over a fairly wide range of 2.5×10¹⁷ to 12×10¹⁷ atoms/cm³ (ASTM '79) could be prepared. In particular, a sample on the low oxygen concentration side which seemed to have not been evaluated very often in the existing techniques could also be prepared.

Doping with nitrogen was performed by preparing a wafer with a nitride film and putting it into a crucible with a silicon raw material and performing melting. The nitrogen doping amount was obtained by calculation from the film thickness of the nitride film and the weight of the wafer. Moreover, since the initial doping amount was known, a nitrogen concentration in a position in which slicing of a sample was performed was calculated by segregation calculation, and the value thus obtained was used as the nitrogen concentration of each sample. As a result, samples whose levels of a nitrogen concentration were 3×10¹³ to 12×10¹³/cm³ were prepared.

By using the above method, a total of 18 samples in which a nitrogen concentration and an oxygen concentration were set were prepared.

On these samples, first, heat treatment performed at 650° C. for 20 minutes was performed as oxygen donor elimination heat treatment, and p/n determination and measurement of resistivity were then performed. The measurement of resistivity was performed by a four-point probe method. A carrier concentration was calculated from the resistivity by using Irvin curve. Moreover, measurement of an oxygen concentration [Oi] was performed by FT-IR by using the same samples.

Next, heat treatment at 1000° C. for 16 hours was performed on these samples, whereby a nitrogen-oxygen donor was eliminated. As for the nitrogen-oxygen donor, in Patent Literature 2, the nitrogen-oxygen donor is treated as a reversible process in which the nitrogen-oxygen donor can be eliminated and generated; in Patent Literature 3, it is described that the nitrogen-oxygen donor grows into an oxygen precipitation nucleus by heat treatment and the nitrogen-oxygen donor is treated as an irreversible process.

Since these descriptions remain to be confirmed, here, 16 hours which is sufficiently longer than the nitrogen-oxygen donor elimination conditions described in Patent Literatures 2 and 3, WO 2009/025337, and so forth was adopted and a condition under which the nitrogen-oxygen donor is reliably eliminated was selected. The measurement of resistivity was performed again after the heat treatment, and a carrier concentration was calculated.

By performing subtraction on the carrier concentration thus calculated and the carrier concentration before the heat treatment, a carrier concentration, difference Δ[n] (/cm³) was calculated.

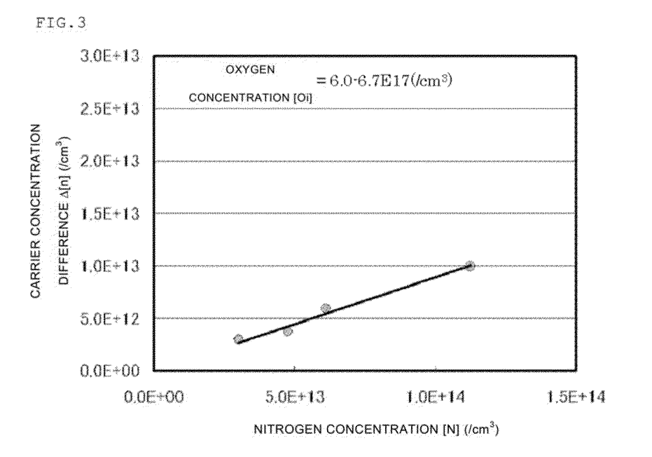

From a total of 18 samples, four levels whose oxygen concentrations are almost the same, the four levels with different nitrogen concentrations, are selected and plotted, whereby FIG. 3 is obtained. The oxygen concentration, range at this time is 6.0×10¹⁷ to 6.7×10¹⁷ atoms/cm³ (ASTM '79).

As is clear from FIG. 3, when the oxygen concentration is at a constant level, the carrier concentration difference Δ[n] (/cm³) is proportional to the nitrogen concentration [N] (/cm³).

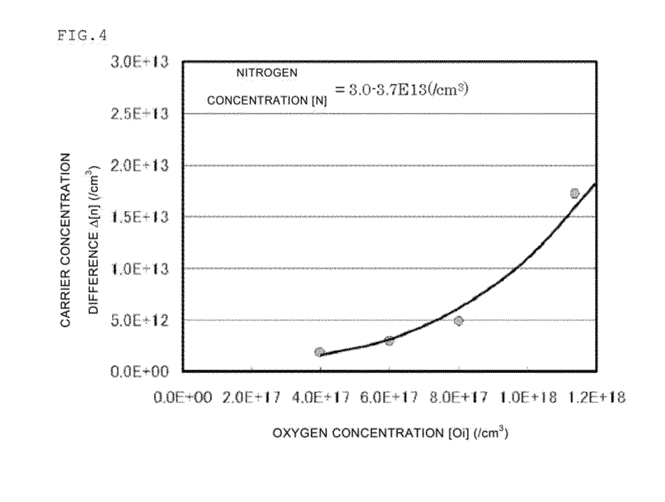

Next, four levels whose nitrogen concentrations are almost the same, the four levels with different oxygen concentrations, are selected and plotted, whereby FIG. 4 is obtained. The nitrogen concentration range at this time is 3.0×10¹³ to 3.7×10¹³/cm³. It is clear from FIG. 4 that, when the nitrogen concentration is at a constant level, the carrier concentration difference Δ[n] (/cm³) depends strongly on the oxygen concentration [Oi] (atoms/cm³(ASTM '79)). The curve in FIG. 4 is depicted by using the third power of the oxygen concentration, and each data is roughly located on the curve.

Based on those described above, if was revealed that, although the carrier concentration caused by the nitrogen-oxygen donor is naturally proportional to the nitrogen concentration, the carrier concentration here is proportional to the third of the oxygen concentration because it is more strongly affected by the oxygen concentration. It is clear that the contribution of the oxygen concentration whose influence has not been made clear sufficiently in the existing techniques is significant.

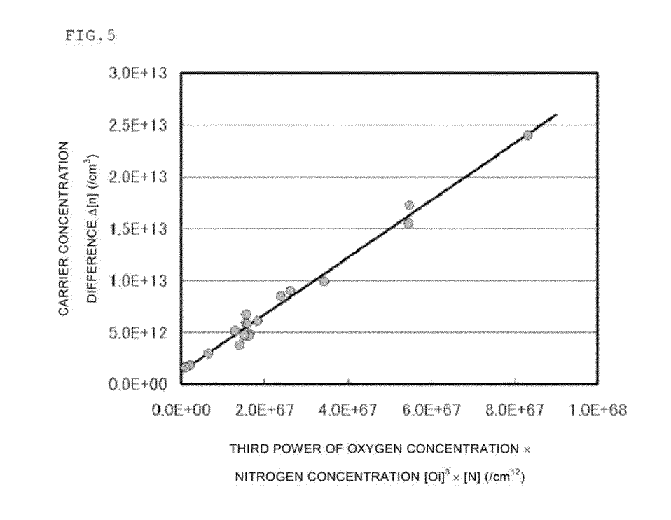

Therefore, by using a total of 18 samples, the carrier concentration differences Δ[n] were further plotted with the product [N]×[Oi]³ of the first power of the nitrogen concentration and the third power of the oxygen concentration on the horizontal axis. The results are depicted in FIG. 5. All of the 18 samples were roughly located on a straight line. An approximate expression (the correlation expression (1)) at this time was expressed as:

[N]=(Δ[n]−1.18×10¹²)/(2.76×10⁻³⁵×[Oi]³.

That is, in the above-described correlation expression [N]=(Δ[n]−β)/α[Oi]³ (where α and β are constants), α=2.76×10⁻⁵⁸ and β=1.18×10¹².

Incidentally, these values of α and β are not universal values and are values obtained as these values under the conditions used in Example 1. These values vary when the measurement conditions and the like change and are not limited to the above values.

Next, the main, test was performed.

A sample obtained by slicing a nitrogen-doped silicon single crystal was prepared as an object to be evaluated of the main test.

By using this sample, first, resistivity after performing heat treatment for oxygen donor elimination, the heat treatment performed at 650° C. for 20 minutes, and resistivity after performing heat treatment for nitrogen-oxygen donor elimination, the heat treatment performed at 1000° C. for 16 hours, were measured by a four-point probe method, and a carrier concentration difference caused by a nitrogen-oxygen donor was obtained. As a result, the carrier concentration difference Δ[n]=7.8×10¹² (/cm³).

On the other hand, the oxygen concentration obtained by FT-IR was [Oi]=8.1×10¹⁷ (atoms/cm³ (ASTM '79)).

When a nitrogen concentration was calculated from these values by using the correlation expression (1) described above, a nitrogen concentration [N]=4.5×10¹³ (/cm³) could be obtained by calculation.

Incidentally, when a production record of a crystal obtained by slicing the object to be evaluated, the object used in the main test, was examined, a target nitrogen concentration in the position of the crystal where the object to be evaluated was taken was 4.3×10¹³ (/cm³).

This value nearly coincides with the value (4.5×10¹³ (/cm³)) of the nitrogen concentration calculated earlier by the method of the present invention.

Therefore, the nitrogen concentration evaluation result using the present invention can be said to be reasonable.

### Comparative Example 1

A sample obtained by slicing a nitrogen-doped silicon single crystal produced by the CZ method was prepared as an object to be evaluated.

By using the object to be evaluated, first, resistivity after performing heat treatment for oxygen donor elimination, the heat treatment performed at 650° C. for 20 minutes, and resistivity after performing heat treatment for nitrogen-oxygen donor elimination, the heat treatment performed at 1000° C. for 16 hours, were measured by a four-point probe method, and a carrier concentration difference caused by a nitrogen-oxygen donor was obtained. As a result, a carrier concentration difference Δ[n]=15.4×10¹² (/cm³).

As described above, since the value (15.4×10¹² (/cm³)) of the measured carrier concentration difference was almost twice the value (7.8×10¹² (/cm³)) of the carrier concentration difference in the object to be evaluated in the main test of Example 1, with no consideration for the influence of the oxygen concentration, the nitrogen concentration was simply estimated to be also twice the nitrogen concentration of the object to be evaluated in Example 1. That is, the nitrogen concentration was estimated to be 6.6×10^{—}(/cm³) which was twice 4.3×10¹³ (/cm³).

Incidentally, when a production record of a crystal obtained by slicing the object to be evaluated was examined, a target nitrogen concentration in the position, of the crystal where the object to be evaluated was taken was 4.3×10¹³ (/cm³). That is, the value is the same as the value of the object to be evaluated of Example 1.

On the other hand, when an oxygen concentration of the object to be evaluated of Comparative Example 1 was measured by FT-IR, the oxygen concentration was [Oi]=10.5×10¹⁷ (atoms/cm³ (ASTM '79)) and was higher than the value in Example 1.

As described above, the reason why, in Comparative Example 1, while, in actuality, the value of the nitrogen concentration was the same as the value of the nitrogen concentration of the object to be evaluated in Example 1, it was estimated that the value was twice the value of the nitrogen concentration of the object to be evaluated in Example 1 is that it was assumed that the nitrogen-oxygen donor was proportional to the nitrogen concentration with no consideration for the oxygen concentration.

This can be said to be an example in which, even when the nitrogen concentrations are the same, if the oxygen concentrations are different, the obtained carrier concentration differences differ greatly even when there is not a large difference in oxygen concentration.

### Example 2

When an object to be evaluated which was similar to the object of Comparative Example 1 was prepared as an object to be evaluated in the main test and a carrier concentration difference and an oxygen concentration were measured, the carrier concentration difference Δ[n]=15.4×10¹² (/cm³) and the oxygen concentration [Oi]=10.5×10¹⁷ (atoms/cm³ (ASTM '79)), and, when a nitrogen concentration was calculated by the correlation expression (1) which was similar to the correlation expression of Example 1, the nitrogen concentration [N]=4.5×10¹³ (/cm³) was obtained.

As described above, since a target nitrogen concentration in the position of the crystal where the object to be evaluated was taken was 4.3×10¹³ (/cm³), unlike Comparative Example 1, nearly-matched results could be obtained.

### Example 3

A sample obtained by slicing a nitrogen-doped silicon single crystal was prepared, as an object to be evaluated in the main test.

By using this sample, first, resistivity after performing heat treatment for oxygen donor elimination, the heat treatment performed at 650° C. for 20 minutes, and resistivity after performing heat treatment for nitrogen-oxygen donor elimination, the heat treatment performed at 1000° C. for 16 hours, were measured by a four-point probe method, and a carrier concentration difference caused by a nitrogen-oxygen donor was obtained. As a result, the carrier concentration difference Δ[n]=8.3×10¹² (/cm³).

On the other hand, the oxygen concentration obtained by FT-IR was [Oi]=4.2×10¹⁷ (atoms/cm³ (ASTM '79)).

When a nitrogen concentration was calculated from these values by using the correlation expression (1) described above, a nitrogen concentration [N]=3.5×10¹⁴ (/cm³) could be obtained by calculation.

Incidentally, when a production record of a crystal obtained by slicing the object to be evaluated, the object used in the main test, was examined, a target nitrogen concentration in the position of the crystal where the object to be evaluated was taken was 3.2×10¹⁴ (/cm³).

This value nearly coincides with the value (3.5×10¹⁴ (/cm³)) of the nitrogen concentration calculated earlier by the method of the present invention.

Therefore, even when the nitrogen concentration was high and the oxygen concentration was low, the appropriateness of the method of the present invention was verified.

### Example 4

A method for calculating a resistivity shift amount in the present invention was performed.

The preliminary test is the same as the preliminary test of Example 1, the same correlation expression (1) can be used, and a modified form thereof is the following correlation expression (1)′.

Δ[n]=2.76×10⁻⁵⁵×[N]×[Oi]³+1.18×10¹²

Next, the main test was performed.

A P-type boron-doped wafer whose target nitrogen concentration [N]=3.5×10¹³ (/cm³) and oxygen concentration [Oi]=10.5×10¹⁷ (atoms/cm³ (ASTM '79)) was prepared.

The resistivity of the wafer after heat treatment for oxygen donor elimination was 156Ω cm. A thermal simulation that mimics a device process was performed on the wafer. This thermal simulation mimics the thermal history at the time of production of a device, and the temperature is 750° C. to 1000° C. and the total treatment time is about 30 hours. Since the maximum temperature is 1000° C., it is estimated that the resistivity changes if there is a nitrogen-oxygen donor.

Therefore, by using the correlation expression (1)′, a carrier concentration difference [n] caused by the nitrogen-oxygen donor was calculated. As a result, the carrier concentration difference Δ[n]=1.3×10¹³ (/cm³) was obtained by calculation.

Since this is a P-type, resistivity after the thermal simulation was calculated from the value obtained by adding the carrier concentration difference to the carrier concentration corresponding to 156Ω cm, As a result, the resistivity was reduced to 135Ω cm, and it was expected that the resistivity shift amount was −21Ω m.

The resistivity of the sample was actually measured again after the thermal simulation. As a result, the resistivity was 138Ω cm and the resistivity shift amount was −18Ω cm. These values nearly coincided with the resistivity (135Ω cm) and the resistivity shift amount (−21Ω cm) which were expected by the present invention before the thermal simulation. Therefore, it can be said that calculation of a resistivity shift amount after heat treatment by the present invention was appropriate.

It is to be understood that the present invention is not limited in any way by the embodiment thereof described, above. The above embodiment is merely an example, and anything that has substantially the same structure as the technical idea recited in the claims of the present invention and that offers similar workings and benefits fails within the technical scope of the present invention.

1-6. (canceled)

7. A method for calculating a nitrogen concentration in a silicon single crystal doped with nitrogen, wherein
a correlation among a carrier concentration difference Δ[n] obtained from a difference between resistivity after heat treatment by which an oxygen donor is eliminated and resistivity after heat treatment by which a nitrogen-oxygen donor is eliminated, an oxygen concentration [Oi], and a nitrogen concentration [N] in the nitrogen-doped silicon single crystal is obtained in advance, and
an unknown nitrogen concentration [N] in a nitrogen-doped silicon single crystal is obtained by calculation from the carrier concentration difference Δ[n] and the oxygen concentration [Oi] based on the correlation.

8. The method for calculating a nitrogen concentration in a silicon single crystal according to claim 7, wherein
when the unknown nitrogen concentration [N] is calculated, calculation is performed by using a correlation expression:
[N]=(Δ[n]−β)/α[Oi]^{2.5 to 3.5}
(where α and β are constants)
from the carrier concentration difference Δ[n] and the oxygen concentration [Oi].

9. The method for calculating a nitrogen concentration in a silicon single crystal according to claim 7, wherein
the nitrogen-doped silicon single crystal is a nitrogen-doped silicon single crystal grown by a Czochralski method.

10. The method for calculating a nitrogen concentration in a silicon single crystal according to claim 8, wherein
the nitrogen-doped silicon single crystal is a nitrogen-doped silicon single crystal grown by a Czochralski method.

11. A method for calculating a resistivity shift amount in a silicon single crystal doped with nitrogen, wherein
a correlation among a carrier concentration difference Δ[n] obtained from a difference between resistivity after heat treatment by which an oxygen donor is eliminated and resistivity after heat treatment by which a nitrogen-oxygen donor is eliminated, an oxygen concentration [Oi], and a nitrogen concentration [N] in the nitrogen-doped silicon single crystal is obtained in advance, and
an unknown carrier concentration difference Δ[n] in a nitrogen-doped silicon single crystal is calculated from the nitrogen concentration [N] and the oxygen concentration [Oi] based on the correlation and a resistivity shift amount by the heat treatment by which the nitrogen-oxygen donor is eliminated is obtained from the calculated carrier concentration difference Δ[n].

12. The method for calculating a resistivity shift amount according to claim 11, wherein
when the unknown carrier concentration difference Δ[n] is calculated, calculation is performed by using a correlation expression:
Δ[n]=α[N]×[Oi]^{2.5 to 3.5}β
(where α and β are constants)
from the nitrogen concentration [N] and the oxygen concentration [Oi].

13. The method for calculating a resistivity shift amount according to claim 11, wherein
the nitrogen-doped silicon single crystal is a nitrogen-doped silicon single crystal grown by a Czochralski method.

14. The method for calculating a resistivity shift amount according to claim 12, wherein
the nitrogen-doped silicon single crystal is a nitrogen-doped silicon single crystal grown by a Czochralski method.
